(19) United States
(12) Patent Application Publication
(10) Pub. No.: US 2021/0290816 A1
(43) Pub. Date: Sep. 23, 2021

(54) LIQUID EMBOLICS

(71) Applicant: MicroVention, Inc., Aliso Viejo, CA (US)

(72) Inventors: Matthew Fitz, Aliso Viejo, CA (US); Gregory M. Cruise, Rancho Santa Margarita, CA (US); Makoto Harumoto, Irvine, CA (US); Yue Wu, Irvine, CA (US)

(21) Appl. No.: 17/204,581

(22) Filed: Mar. 17, 2021

Related U.S. Application Data
(60) Provisional application No. 62/990,812, filed on Mar. 17, 2020.

(51) Int. Cl. A61L 24/00; A61K 31/337; A61K 31/4745; A61K 31/351; A61K 31/404; A61K 31/44; A61K 31/7068; A61K 31/282; A61K 31/664; A61K 31/4188; A61K 31/17; A61K 51/06; A61L 24/04

(52) U.S. Cl. CPC A61L 24/0015; A61L 2300/44; A61K 31/4745; A61K 31/351; A61K 31/404; A61K 31/44; A61K 31/7068; A61K 31/282; A61K 31/664; A61K 31/4188; A61K 31/17; A61K 51/06; A61L 24/046; A61L 2300/204; A61L 2300/216; A61L 2300/224; A61K 31/337

(57) ABSTRACT

Described herein are formulations that transition from a liquid state to a solid state for use in the embolization of arteriovenous malformations (AVM's) and solid tumors.

### CROSS REFERENCE TO RELATED APPLICATION

This application claims the benefit of U.S. provisional patent application No. 62/990,812, filed Mar. 17, 2020, the entire disclosure of which is incorporated herein by reference.

### FIELD

Described herein are medical treatment methods, and more particularly to solutions that transition from a liquid to a solid for use in the embolization of arteriovenous malformations (AVM's) and solid tumors.

### BACKGROUND

Liquid embolics are introduced through a microcatheter in the liquid state and transition to the solid state once in the body. The transition is generally controlled either by reaction or precipitation. For the materials functioning by reaction, the materials are introduced in a liquid state and undergo a chemical reaction to transition to a solid state. In some embolics, a pharmaceutical drug or therapeutic agent is dissolved in one of the two parts that are combined to form the solid liquid embolic. For the materials functioning by precipitation, the materials are introduced in a non-physiological condition and transition to a solid upon exposure to physiological conditions. Non-physiological conditions include water miscible organic solvents, temperature, and pH.

The liquid embolics functioning by precipitation have been widely investigated. Precipitation from water miscible organic solvents has been utilized to control the transition from a liquid state to a solid state. Some examples provide a water insoluble polymer, ethylene vinyl acetate, used in conjunction with a water miscible organic solvent, dimethyl sulfoxide. Other examples provide an intrinsically radiopaque, water-insoluble polymer used in conjunction with a water miscible organic solvent, dimethyl sulfoxide. Still other examples provide an alternative intrinsically radiopaque, water-insoluble polymer used in conjunction with a water miscible organic solvent, dimethyl sulfoxide. Upon exposure to blood, all three polymers precipitate from the water miscible organic solvent and form an insoluble mass to occlude blood flow.

Radioactivity enhances functionality of liquid embolics. Liquid embolics are designed to occlude blood flow in an effort to destroy non-desired tissues such as AVM's and solid tumors. Radioactivity can destroy tissue. For example, some radioactive isotope coated stents are provided to supplement the mechanical support of the stent with a mechanism to destroy the arterial plaque. Radioactivity has also been investigated in conjunction with liquid embolics. Other examples provide a water insoluble polymer, ethylene vinyl acetate, supplemented with a water insoluble radioisotope used in conjunction with a water miscible organic solvent, dimethyl sulfoxide.

### SUMMARY

In some embodiments, liquid embolics are described that are intrinsically radiopaque, available in radiostable and radioactive forms, and deliver pharmaceutical drugs or therapeutic agents to a vascular site.

In some embodiments, liquid embolic solutions or formulations are described that can be deployed into the vasculature using standard practices and microcatheters/catheters to occlude blood flow. In some embodiments, the liquid embolic formulations include a biocompatible polymer with biostable or biodegradable linkages to aromatic rings containing a plurality of iodine atoms (radiostable and/or radioactive) and a water miscible, non-aqueous solvent that dissolves the biocompatible polymer and contains a pharmaceutical drug or therapeutic agent.

In one embodiment, the biodegradable linkage is susceptible to breakage via hydrolysis. In another embodiment, the biodegradable linkage is susceptible to breakage via enzymatic action. In another embodiment, the linkage is biostable.

In one embodiment, described herein are embolic compositions including a substantially stable biocompatible polymer comprising a reaction product of a first monomer including a polymerizable moiety having a biodegradable or biostable linkage to a visualization agent having at least one aromatic ring including at least one iodine atom, and a second monomer including a polymerizable moiety and at least one hydroxyl group; and a non-physiological solution containing a pharmaceutical drug or therapeutic agent. In some embodiments, the substantially stable biocompatible polymer is soluble in the non-physiological solution and insoluble in a physiological solution.

In some embodiments, at least one of the iodine atom included in the herein described liquid embolic is a radioactive isotope. The radioactive isotope can be ¹²³I, ¹²⁴I, ¹²⁵I, ¹³¹I, or a combination thereof.

In one embodiment, the stable or radiostable iodine isotope is ¹²⁷I and the radioactive iodine isotope is ¹²³I, ¹²⁴I, ¹²⁵I, or ¹³¹I.

In one embodiment, the pharmaceutical drug or therapeutic agent is doxorubicin, irinotecan, sunitinib, sorafenib, paclitaxel, temozolomide, carmustine, cyclophosphamide, vinchristine, and/or an antibody.

Methods of treating are also described. In one embodiment, methods of treating can comprise delivering an embolic composition as described herein to a treatment site. In some embodiments, the delivering results in the substantially stable biocompatible polymer precipitating in the physiological solution. The treatment site can be within a lumen, such as but not limited to a blood vessel.

### BRIEF DESCRIPTION OF THE DRAWINGS

FIG. 1 illustrates kinetics of paclitaxel elution from a liquid embolic solution.

FIG. 2 illustrates kinetics of irinotecan elution from a liquid embolic solution.

FIG. 3 illustrates kinetics of doxorubicin elution from a liquid embolic solution.

FIG. 4 illustrates kinetics of sunitinib elution from a liquid embolic solution.

FIG. 5 illustrates kinetics of sorafenib elution from a liquid embolic solution.

FIG. 6 illustrates kinetics of gemcitabine elution from a liquid embolic solution.

FIG. 7 illustrates kinetics of oxaliplatin elution from a liquid embolic solution.

FIG. 8 illustrates kinetics of cyclophosphamide elution from a liquid embolic solution.

FIG. 9 illustrates kinetics of temozolomide elution from a liquid embolic solution.

FIG. 10 illustrates kinetics of carmustine elution from a liquid embolic solution.

FIG. 11 illustrates a post-embolization angiography demonstrated excellent penetration into the distal liver vasculature.

FIG. 12 illustrates quantification of irinotecan in the blood showing a rapid rise with tapering back to baseline beyond the timescale of the experiment.

FIG. 13 illustrates a post-embolization angiography demonstrated excellent penetration into the distal liver vasculature.

FIG. 14 illustrates quantification of doxorubicin in the blood showing a rapid rise with tapering back to baseline.

FIG. 15 illustrates a post-embolization angiography demonstrated excellent penetration into the distal liver vasculature.

FIG. 16 illustrates quantification of oxaliplatin in the blood showing a rapid rise with tapering back to baseline beyond the timescale of the experiment.

### DETAILED DESCRIPTION

Described herein are medical treatment solutions that transition from a liquid state to a solid state for use in the embolization of arteriovenous malformations (AVM's) and solid tumors. Methods of using these solutions are also described. Some embodiments described herein include a biocompatible polymer with one or more covalently bound iodine isotopes (radiostable and/or radioactive) and a non-physiological solution containing a pharmaceutical drug or therapeutic agent. The precipitation of the liquid embolic in a vascular defect can induce stasis of the blood, and subsequently the pharmaceutical drug can be delivered from the solid liquid embolic to the surrounding tissue with reduced washout.

Delivery of pharmaceutical drugs or therapeutic agents can be an addition to the capabilities of liquid embolics. In some embodiments, embolics with pharmaceutical drugs or therapeutic agents can be used in cases where an objective is to eliminate vasculature and/or tissue like AVM's and hypervascular tumors. The addition of a pharmaceutical drug or therapeutic agent to the stasis of the blood flow can be induced by the solid liquid embolic could further the effectiveness of liquid embolics in the treatment of vascular diseases.

The liquid embolics described herein can comprise (i) a biocompatible polymer with an aromatic ring with a plurality of iodine atoms coupled via biodegradable or biostable linkages and (ii) a water miscible solvent that dissolves the biocompatible polymer and dissolves or suspends the pharmaceutical drug or therapeutic agent.

In some embodiments, the liquid embolic described herein can be a precipitating hydrophobic injectable liquid (PHIL®, MicroVention, Inc., Aliso Viejo, Calif.). In some embodiments, this embolic composition includes an iodine based contrast bonded to the polymer to make it radio opaque.

The main function of the liquid embolic polymer can be to solidify in the vasculature or other anatomical structure when coming in contact with blood or other physiological fluid to occlude the vessel or structure and to permit visualization of the polymer when imaged using medically relevant techniques. The liquid embolic polymer's solubility can be achieved with the judicious selection of the composition of the polymer to ensure that it is essentially insoluble at physiological conditions. In some embodiments, the liquid embolic polymer is prepared from monomers containing visualization species and optionally other monomers. The ratio of monomers with monomers containing visualization species and other monomers can be dependent on the structure of the monomers.

The monomer or monomers with visualization species can impart visibility of the liquid embolic polymer when imaged using a medically relevant imaging technique such as fluoroscopy, or computed tomography (CT). Characteristic features of the monomers with visualization species can be cores that are visible under medically relevant imaging techniques and one or more polymerizable moiety attached to the core with a biodegradable linkage.

Visualization of the polymer under fluoroscopy and CT imaging can be imparted by the use of monomers with cores containing iodine, particularly aromatic rings with a plurality of iodine atoms. A preferred core containing iodine is triiodophenol. Concentrations of iodine to render the liquid embolic visible using fluoroscopy or CT imaging can range from about 20% w/w to about 50% w/w of the liquid embolic solution.

In some embodiments, polymerizable moieties are those that permit free radical polymerization, including acrylates, methacrylates, acrylamides, methacrylamides, vinyl groups, and derivatives thereof. Alternatively, other reactive chemistries can be employed to polymerize the liquid embolic polymer, such as, but not limited to, nucleophile/N-hydroxysuccinimde esters, nucleophile/halide, vinyl sulfone/acrylate or maleimide/acrylate. In one embodiment, polymerizable moieties are acrylates and acrylamides.

Biodegradable linkages permit the separation of a visualization core from the polymer. After separating from the polymer, the core is removed by diffusion or cells comprising the foreign body response to the polymer. Biodegradable linkages can be separated into two types, those susceptible to hydrolysis and those susceptible to enzymatic action. Linkages susceptible to hydrolysis are generally esters or polyesters. Esters can be introduced by reacting hydroxyl groups with strained anhydrides, such as succinic or glutaric anhydride, or cyclic esters, such as lactide, glycolide, ε-caprolactone, and trimethylene carbonate. The rate of degradation can be controlled by the selection of the ester and the number of the esters inserted into the biodegradable linkages. Linkages susceptible to enzymatic action are generally peptides that are degraded by particular enzymes, such as matrix metalloproteinases, collagenases, elastases, cathepsin. Peptide sequences degraded by matrix metalloproteinases include Gly-Pro-Gln-Gly-Ile-Ala-Ser-Gln, Gly-Pro-Gln-Gly\Pro-Ala-Gly-Gln, Lys-Pro-Leu-Gly-Leu-Lys-Ala-Arg-Lys, Gly-Pro-Gln-Ile-Trp-Gly-Gln, and Gln-Pro-Gln-Gly-Leu-Ala-Lys. Peptide sequences degraded by cathepsin include Gly-Phe-Gln-Gly-Val-Gln-Phe-Ala-Gly-Phe, Gly-Phe-Gly-Ser-Val-Gln-Phe-Ala-Gly-Phe, and Gly-Phe-Gly-Ser-Thr-Phe-Phe-Ala-Gly-Phe. Peptide sequences degraded by collagenase include Gly-Gly-Leu-Gly-Pro-Ala-Gly-Gly-Lys and Ala-Pro-Gly-Leu. Peptide sequences degraded by papain include Gly-Phe-Leu-Gly. Peptide sequences degraded by caspase-3 include Asp-Glu-Val-Asp-Thr. The rate of degradation can be controlled by the peptide sequence selection.

Other monomers can contain a polymerizable moiety and have a structure that is conducive to the desired solubility characteristics. Preferred polymerizable moieties can be those that permit free radical polymerization, including acrylates, methacrylates, acrylamides, methacrylamides, vinyl groups, and derivatives thereof. Alternatively, other reactive chemistries can be employed to polymerize the liquid embolic polymer, such as but not limited to nucleophile/N-hydroxysuccinimde esters, nucleophile/halide, vinyl sulfone/acrylate or maleimide/acrylate. In one embodiment, polymerizable moieties are acrylates and acrylamides. In some embodiments, the other monomer can compensate for the monomers with visualization species. If a prepared polymer is too hydrophobic for dissolution in water miscible solvent, more hydrophilic monomers can be introduced to alter the solubility. If a prepared polymer is too hydrophilic and is soluble in water, more hydrophobic monomers can be introduced to alter the solubility. Other monomers include hydroxyethyl methacrylate, t-butyl acrylate, t-butyl acrylamide, n-octyl methacrylate, and methyl methacrylate.

In some embodiments, the liquid embolic polymers are polymerized from solutions including monomers as described herein comprising visualization species and optionally other monomers. The solvent used to dissolve the monomers can be any solvent that dissolves the desired monomers. In some embodiments, solvents can be aqueous, nonaqueous, or water miscible. In some embodiments, the solvent can include methanol and/or acetonitrile.

Polymerization initiators can be used to start polymerization of monomers in a solution. The polymerization can be initiated by reduction-oxidation, radiation, heat, or any other method known in the art. Radiation cross-linking of the prepolymer solution can be achieved with ultraviolet light or visible light with suitable initiators or ionizing radiation (e.g. electron beam or gamma ray) without initiators. Polymerization can be achieved by application of heat, either by conventionally heating the solution using a heat source such as a heating well, or by application of infrared light to the prepolymer solution.

In one embodiment, the polymerization initiator is azobisisobutyronitrile (AIBN) or a water soluble AIBN derivative (2,2′-azobis(2-methylpropionamidine) dihydrochloride). Other initiators can include AIBN derivatives, including, but not limited to 4,4′-azobis(4-cyanovaleric acid, and other initiators such as N,N,N′,N′-tetramethylethylenediamine, ammonium persulfate, benzoyl peroxides, and combinations thereof, including azobisisobutyronitriles. In some embodiments, initiator concentrations are less than 0.5% w/w of the prepolymer solution. The polymerization reaction can be run at elevated temperatures, such as, but not limited to about 80 degrees Celsius. After the polymerization is completed, the liquid embolic polymer can be recovered by precipitation in a non-solvent and dried under vacuum.

The substitution of radioactive iodine for stable iodine can be performed at any of the steps in the synthetic procedure. In one embodiment, this step can be performed after the conclusion of the preparation of the liquid embolic polymer. After the liquid embolic polymer has been prepared, it is re-dissolved in dimethyl sulfoxide and the sodium salt of the radioactive iodine is added. After the sodium salt has been dissolved (e.g., completely dissolved), 30% hydrogen peroxide in water is added. The reaction solution can be optionally heated to facilitate the substitution. When the reaction is complete, the liquid embolic polymer is purified with repeated precipitation in water and dissolution in dimethyl sulfoxide. Alternatively, the substitution can be performed on the monomer containing a polymerizable moiety with a biostable or biodegradable linkage to an aromatic ring containing a plurality of iodine atoms. The same reaction procedure as described for the liquid embolic polymer may be used for the monomer.

In some embodiments, iodine radioisotopes can include ¹²³I, ¹²⁴I, ¹²⁵I, ¹³¹I, or a combination thereof. Each isotope has distinct properties that ablate tissue and permit imaging. In one embodiment, the isotope is ¹³¹I due to its destructive beta emissions, gamma emissions that can be used for medical imaging, and short half-life.

The water-miscible, non-aqueous solvent can be used to dissolve the liquid embolic polymer and to dissolve or suspend the pharmaceutical drug or therapeutic agent. Concentrations of the liquid embolic polymer in the aqueous solution can range from about 2.5% to about 25%, about 5% to about 15%, or about 2.5% to about 10%.

In one embodiment, a method of preparing the liquid embolic can include dissolving the liquid embolic polymer in a water-miscible, non-aqueous solvent and adding to a syringe, vial or other container. Sterilization before use can be achieved by autoclaving or using gamma irradiation. The pharmaceutical drug or therapeutic agent can be added before sterilization in the manufacturing process or immediately before use by reconstitution in the liquid embolic solution.

In one embodiment, the liquid embolic solvent is dimethyl sulfoxide.

The pharmaceutical drug or therapeutic agent can be any chemical that can be dissolved or suspended in the liquid embolic solution. In one embodiment, pharmaceutical drugs and therapeutic agents can be those used to treat cancer. Cancer treating drugs can include, but are not limited to Abemaciclib, Abiraterone Acetate, Abraxane (Paclitaxel Albumin-stabilized Nanoparticle Formulation), ABVD, ABVE, ABVE-PC, AC, Acalabrutinib, AC-T, Actemra (Tocilizumab), Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Ilydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alpelisib, Alunbrig (Brigatinib), Ameluz (Aminolevuiinic Acid Hydrochloride), Amifostine, Aminolevulinic Acid Hydrochloride, Anastrozole, Apalutamide, Aprepitant, Aranesp (Darbepoetin Alfa), Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Asparlas (Calaspargase Pegol-mknl), Atezolizumab, Avastin (Bevacizumab), Avelumab, Axicabtagene Ciloleucel, Axitinib, Azacitidine, Azedra (Iobenguane I131), Balversa (Erdafitinib), Bavencio (Avelumab), BEACOPP, Beleodaq (Belinostat), Belinostat, Bendamustine Hydrochloride, Bendeka (Bendarnustine Hydrochloride), BEP, Besponsa (Inotuzumab Ozogamicin), Bevacizumab, Bexarotene, Bicalutamide, BiCNU (Carmustine), Binimetinib, Bleomycin Sulfate, Blinatumomab, Blincyto (Blinatumomab), Bortezomib, Bosulif (Bosutinib), Bosutinib, Braftovi (Encorafenib), Brentuximab Vedotin, Brigatinib, Brukinsa (Zanubrutinib), BuMel, Busulfan, Busulfex (Busulfan), Cabazitaxel, Cablivi (Caplacizumab-yhdp), Cabometyx (Cabozantinib-5-Malate), Cabozantinib-5-Malate, CAF, Calaspargase Pegol-mknl, Calquence (Acalabrutinib), Campath (Alemtuzumab), Camptosar (Irinotecan Hydrochloride), Capecitabine, Caplacizumab-yhdp, CAPDX, Carac (Fluorouracil-Topical), Carboplatin, CARBOPLATIN-TAXOL, Carfilzomib, Carmustine, Carmustine Implant, Casodex (Bicalutamide), CEM, Cemiplimab-rwlc, Ceritinib, Cerubidine (Daunorubicin Hydrochloride), Cervarix (Recombinant HPV Bivalent Vaccine), Cetuxirnab, CEV, Chlorambucil, CHLORAMBUCIL-PREDNISONE, CHOP, Cisplatin, Cladribine, Clofarabine, Clolar (Clofarabine), CMF, Cobimetinib, Cometriq (Cabozantinib-S-Malate), Copanlisib Hydrochloride, COPDAC, Copiktra (Duvelisib), COPP, COPP-ABV, Cosmegen (Dactinomycin), Cotellic (Cobimetinib), Crizotinib, CVP, Cyclophosphamide, Cyramza (Ramucirumab), Cytarabine, Dabrafenib Mesylate, Dacarbazine, Dacogen (Decitabine), Dacornitinib, Dactinomycin, Daratumumab, Darbepoetin Alfa, Darolutamide, Darzalex (Daratumumab), Dasatinib, Daunorubicin Hydrochloride, Daunorubicin Hydrochloride and Cytarabine Liposome, Daurismo (Glasdegib Maleate), Decitabine, Defibrotide Sodium, Defitelio (Defibrotide Sodium), Degarelix, Denileukin Diftitox, Denosumab, Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Durvalumab, Duvelisib, Efudex (Fluorouracil-Topical), Eligard (I.euprolide Acetate), Elitek (Rasburicase), Ellence (Epirubicin Hydrochloride), Elotuzumab, Eloxatin (Oxaliplatin), Eltrombopag Olamine, Elzonris (Tagraxofusp-erzs), Emapalumab-lzsg, Emend (Aprepitant), Empliciti (Elotuzumab), Enasidenib Mesylate, Encorafenib, Entrectinib, Enzalutamide, Epirubicin Hydrochloride, EPOCH, Epoetin Alfa, Epogen (Epoetin Alfa), Erbitux (Cetuximab), Erdafitinib, Eribulin Mesylate, Erivedge (Vismodegib), Erleada (Apalutamide), Erlotinib Hydrochloride, Erwinaze (Asparaginase Erwinia chrysanthemi), Ethyol (Amifostine), Etopophos (Etoposide Phosphate), Etoposide, Etoposide Phosphate, Everolimus, Evista (Raloxifene Hydrochloride), Evomela (Melphalan Hydrochloride), Exemestane, 5-FU (Fluorouracil Injection), 5-FU (Fluorouracil-Topical), Fareston (Toremifene), Farydak (Panobinostat), Faslodex (Fulvestrant), FEC, Fedratinib Hydrochloride, Femara (Letrozole), Filgrastim, Firmagon (Degarelix), Fludarabine Phosphate, Fluoroplex (Fluorouracil-Topical), Fluorouracil Injection, Fluorouracil-Topical, Flutamide, FOLFIRI, FOLFIRI-8 EVACIZU MAB, FOLFIRI-CETUXIMAB, FOLFIRINOX, FOLFOX, Folotyn (Pralatrexate), Fostarnatinib Disodium, FU-LV, Fulvestrant, Gamifant (Emapalumab-lzsg), Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gilteritinib Fumarate, Glasdegib Maleate, Gleevec (Imatinib Mesylate), Gliadel Wafer (Carmustine Implant), Glucarpidase, Goserelin Acetate, Granisetron, Granisetron Hydrochloride, Granix (Filgrastim), Halaven (Eribulin Mesylate), Hemangeol (Propranolol Hydrochloride), Herceptin Hylecta (Trastuzumab and Hyaluronidase-oysk), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin PFS (Idarubicin Hydrochloride), Idarubicin Hydrochloride, Idelalisib, Idhifa (Enasidenib Mesylate), Ifex (Ifosfamide), Ifosfamide, II-2 (Aldesleukin), Imatinib Mesylate, Imbruvica (Ibrutinib), Imfinzi (Durvalumab), Imiquimod, Imlygic (Talimogene laherparepvec), Inlyta (Axitinib), Inotuzumab Ozogamicin, Inrebic (Fedratinib Hydrochloride), Interferon Alfa-2b, Recombinant, Interleukin-2 (Aldesleukin), Intron A (Recombinant Interferon Alfa-2b), Iobenguane I131, Ipilimumab, Iressa (Gefitinib), Irinotecan Hydrochloride, Irinotecan Hydrochloride Liposome, Istodax (Romidepsin), Ivosidenib, Ixabepilone, Ixazomib Citrate, Ixempra (Ixabepilone), Jakafi (Ruxolitinib Phosphate), JES, Jevtana (Cabazitaxel), Kadcyla (Ado-Trastuzumab Emtansine), Kepivance (Palifermin), Keytruda (Pembrolizumab), Kisqali (Ribociclib), Kymriah (Tisagenlecleucel), Kyprolis Lanreotide Acetate, Lapatinib Ditosylate, Larotrectinib Sulfate, Lenalidomide, Lenvatinib Mesylate, Lenvima (Lenvatinib Mesylate), Letrozole, Leucovorin Calcium, Leukeran (Chlorambucil), Leuprolide Acetate, Levulan Kerastik (Aminolevulinic Acid Hydrochloride), Libtayo (Cemiplimab-rwlc), Lomustine, Lonsurf (Trifluridine and Tipiracil Hydrochloride), Lorbrena (Lorlatinib), Lorlatinib, Lumoxiti (Moxetumomab Pasudotox-tdfk), Lupron (Leuprolide Acetate), Lupron Depot (Leuprolide Acetate), Lutathera (Lutetium Lu 177-Dotatate), Lutetium (Lu 177-Dotatate), Lynparza (Olaparib) (Carfilzomib), Marqibo (Vincristine Sulfate Liposome), Matulane (Procarbazine Hydrochloride), Mechlorethamine Hydrochloride, Megestrol Acetate, Mekinist (Trametinib), Mektovi (Binimetinib), Melphalan, Melphalan Hydrochloride, Mercaptopurine, Mesna, Mesnex (Mesna), Methotrexate, Methylnaltrexone Bromide, Midostaurin, Mitomycin C, Mitoxantrone Hydrochloride, Mogamulizumab-kpkc, Moxetumomab Pasudotox-tdfk, Mozobil (Plerixafor), Mustargen (Mechloretharnine Hydrochloride), MVAC, Mvasi (Bevacizumab), Myleran (Busulfan), Mylotarg (Gerntuzumab Ozogamicin), Nanoparticle Paclitaxel (Pacitaxel Albumin-stabilized Nanoparticle Formulation), Navelbine (Vinorelbine Tartrate), Neciturnurnab, Nelarabine, Neratinib Maleate, Nerlynx (Neratinib Maleate), Netupitant and Palonosetron Hydrochloride, Neulasta (Pegfilgrastim), Neupogen (Filgrastirn), Nexavar (Sorafenib Tosylate), Nilandron (Nilutarnide), Nilotinib, Nilutamide, Ninlaro (Ixazomib Citrate), Niraparib Tosylate Monohydrate, Nivolurnab, Nplate (Romiplostim), Nubeqa (Darolutamide), Obinutuzumab, Odomzo (Sonidegib), OEPA, Ofatumumab, OFF, Ola pa rib, Omacetaxine Mepesuccinate, Oncaspar (Pegaspargase), Ondansetron Hydrochloride, Onivyde (Irinotecan Hydrochloride Uposome), Ontak (Denileukin Diftitox), Opdivo (Nivolumab), OPPA, Osimertinib Mesylate, Oxaliplatin, Paclitaxel, Paclitaxel Albumin-stabilized Nanoparticle Formulation, PAD, Palbociclib, Palifermin, Paionosetron Hydrochloride, Palonosetron Hydrochloride and Netupitant, Parnidronate Disodium, Paniturnumab, Panobinostat, Pazopanib Hydrochloride, PCV, PEB, Pegaspargase, Pegfilgrastim, Peginterferon Aifa-2b, PEG-Intron (Peginterferon Alfa-2b), Pernbrolizumab, Pemetrexed Disodium, Perjeta (Pertuzumab), Pertuzumab, Piqray (Alpelisib), Plerixafor, Polatuzumab Vedotin-piiq, Polivy (Polatuzumab Vedotin-piiq), Pomalidomide, Pomalyst (Pomalidomide), Ponatinib Hydrochloride, Portrazza (Necitumumab), Poteligeo (Mogamulizumab-kpkc), Pralatrexate, Prednisone, Procarbazine Hydrochloride, Procrit (Epoetin Alfa), Proleukin (Aldesleukin), Prolia (Denosumab), Promacta (Eltrombopag Olamine), Propranolol Hydrochloride, Provenge (Sipuleucei-T), Purinethol (Mercaptopurine), Purixan (Mercaptopurine), Radium 223 Dichloride, Raloxifene Hydrochloride, Rarnucirumab, Rasburicase, Ravulizumab-cwvz, R-CHOP, R-CVP, Recombinant Human Papiilomavirus (HPV) Bivalent Vaccine, Recombinant Human Papillomavirus (HPV) Nonavalent Vaccine, Recombinant Human Papillomavirus (HPV} Quadrivalent Vaccine, Recombinant Interferon Alfa-2b, Regorafenib, Relistor (Methylnaltrexone Bromide), R-EPOCH, Retacrit (Epoetin Alfa), Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and Hyaluronidase Human, Rolapitant Hydrochloride, Romidepsin, Romiplostim, Rozlytrek (Entrectinib), Rubidomycin (Daunorubicin Hydrochloride), Rubraca (Rucaparib Camsylate), Rucaparib Camsylate, Ruxoiitinib Phosphate, Rydapt (Midostaurin), Sancuso (Granisetron), Sclerosol Intrapleural Aerosol (Talc), Selinexor, Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sustol (Granisetron), Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib Mesylate), Tag raxofusp-erzs, Tagrisso (Osimertinib Mesylate), Talazoparib Tosylate, Talc, Talimogene Laherparepvec, Talzenna (Talazoparib Tosylate), Tamoxifen Citrate, Tarceva (Erlotinib Hydrochloride), Targretin (Bexarotene), Tasigna (Nilotinib), Tavalisse (Fostamatinib Disodium), Taxol (Paclitaxel), Taxotere (Docetaxel), Tecentriq (Atezolizumab), Temodar (Temozolomide), Temozolomide, Temsirolimus, Thalidomide, Thalomid (Thalidomide), Thioguanine, Thiotepa, Tibsovo (Ivosidenib), Tisagenlecleucel, Tocilizumab, Tolak (Fluorouracil-Topicai), Topotecan Hydrochloride, Toremifene, Torisel (Temsirolimus), Totect (Dexrazoxane Hydrochloride), TPF, Trabectedin, Trarnetinib, Trastuzumab, Trastuzurnab and Hyaluronidase-oysk, Treanda (Bendarnustine Hydrochloride), Trexall (Methotrexate), Trifluridine and Tipiracil Hydrochloride, Trisenox (Arsenic Trioxide), Truxima (Rituximab), Tykerb (Lapatinib Ditosylate), Ultomiris (Ravulizumab-cwvz), Unituxin (Dinutuximab), Uridine Triacetate, VAC, Valrubicin, Valstar (Valrubicin), Vandetanib, VAMP, Varubi (Rolapitant Hydrochloride), Vectibix (Panitumumab), VeIP, Velcade (Bortezornib), Vemurafenib, Venclexta (Venetoclax), Venetoclax, Verzenio (Abemaciclib), Vidaza (Azacitidine), Vinblastine Sulfate, Vincristine Sulfate, Vincristine Sulfate Uposome, Vinorelbine Tartrate, VIP, Vismodegib, Vistogard (Uridine Triacetate), Vitrakvi (Larotrectinib Sulfate), Vizimpro (Dacomitinib), Voraxaze (Glucarpidase), Vorinostat, Votrient (Pazopanib Hydrochloride), Vyxeos (Daunorubicin Hydrochloride and Cytarabine Liposome), Xalkori (Crizotinib), Xeloda (Capecitabine), XEURI, XELOX, Xgeva (Denosumab), Xofigo (Radium 223 Dichloride), Xospata (Gilteritinib Fumarate), Xpovio (Selinexor), Xtandi (Enzalutamide), Yervoy (Ipilimumab), Yescarta (Axicabtagene Ciloleucel), Yondelis (Trabectedin), Zaltrap (Ziv-Aflibercept), Zanubrutinib, Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zoli nza (Vorinostat), Zometa (Zoledronic Acid), Zydelig (Idelalisib), Zykadia (Ceritinib), Zytiga (Abiraterone Acetate), and combinations thereof.

Additionally, pharmaceutical drugs and therapeutic agents not related to the treatment of cancer may be incorporated into the liquid embolics. These pharmaceutical drugs and therapeutic agents can include, but are not limited to anti-angiogenic factors, anti-inflammatory drugs, analgesics, anti-coagulation agents, coagulation agents, clotting agents, local anesthetics, and the like. Combinations of any of the pharmaceutical drugs and therapeutic agents described can be used.

In some embodiments, the herein described embolic formulations can deliver the pharmaceutical drug or therapeutic agent at a particular rate or by a particular release profile. In some embodiments, that release profile can be first order, second order, third order or the like. In some embodiments, the profile can be rapid release followed by a plateaued steady release.

In some embodiments, the particular drug or therapeutic agent can be a logarithmic or about a logarithmic curve with a sharp increase over a first time period followed by a plateau thereafter during a second time period.

In some embodiments, the first time period is about 90 min, about 80 min, about 70 min, about 65 min, about 60 min, between about 90 min and about 60 min, between about 80 min and about 60 min, between about 90 min and about 80 min, between about 70 min and about 60 min, or between about 80 min and about 70 min.

In some embodiments, the second time period is about 2 min, about 3 min, about 4 min, about 5 min, about 6 min, about 7 min, about 8 min, about 9 min, about 10 min, about 11 min, about 12 min , about 13 min, about 14 min, about 15 min, about 20 min, about 25 min, about 30 min, about 35 min, about 40 min, about 50 min, between about 2 min and about 50 min, between about 2 min and about 10 min, between about 20 min and about 50 min, between about 2 min and about 5 min, or between about 5 min and about 10 min.

In one embodiment, the pharmaceutical drug or therapeutic agent is paclitaxel.

In one embodiment, paclitaxel is released or eluted from the liquid embolic at a logarithmic rate. In one embodiment, that rate is over about 90 min. In some embodiments, paclitaxel is exponentially released over the first about 9 min.

In one embodiment, the pharmaceutical drug or therapeutic agent is irinotecan.

In one embodiment, irinotecan is released or eluted from the liquid embolic at a logarithmic rate. In one embodiment, that rate is over about 90 min. In some embodiments, irinotecan is exponentially released over the first about 9 min.

In one embodiment, the pharmaceutical drug or therapeutic agent is doxorubicin.

In one embodiment, doxorubicin is released or eluted from the liquid embolic at a logarithmic rate. In one embodiment, that rate is over about 90 min. In some embodiments, doxorubicin is exponentially released over the first about 14 min.

In one embodiment, the pharmaceutical drug or therapeutic agent is sunitinib.

In one embodiment, sunitinib is released or eluted from the liquid embolic at a logarithmic rate. In one embodiment, that rate is over about 65 min. In some embodiments, sunitinib is exponentially released over the first about 2 min.

In one embodiment, the pharmaceutical drug or therapeutic agent is sorafenib.

In one embodiment, sorafenib is released or eluted from the liquid embolic at a logarithmic rate. In one embodiment, that rate is over about 90 min. In some embodiments, sorafenib is exponentially released over the first about 2 min.

In one embodiment, the pharmaceutical drug or therapeutic agent is gemcitabine.

In one embodiment, gemcitabine is released or eluted from the liquid embolic at a logarithmic rate. In one embodiment, that rate is over about 90 min. In some embodiments, gemcitabine is exponentially released over the first about 9 min.

In one embodiment, the pharmaceutical drug or therapeutic agent is oxaliplatin.

In one embodiment, oxaliplatin is released or eluted from the liquid embolic at a logarithmic rate. In one embodiment, that rate is over about 80 min. In some embodiments, oxaliplatin is exponentially released over the first about 15 min.

In one embodiment, the pharmaceutical drug or therapeutic agent is cyclophosphamide.

In one embodiment, cyclophosphamide is released or eluted from the liquid embolic at a logarithmic rate. In one embodiment, that rate is over about 90 min. In some embodiments, cyclophosphamide is exponentially released over the first about 35 min.

In one embodiment, the pharmaceutical drug or therapeutic agent is temozolomide.

In one embodiment, temozolomide is released or eluted from the liquid embolic at a logarithmic rate. In one embodiment, that rate is over about 90 min. In some embodiments, temozolomide is exponentially released over the first about 35 min.

In one embodiment, the pharmaceutical drug or therapeutic agent is carmustine.

In one embodiment, carmustine is released or eluted from the liquid embolic at a logarithmic rate. In one embodiment, that rate is over about 90 min. In some embodiments, carmustine is exponentially released over the first about 35 min.

In one embodiment, the pharmaceutical drug or therapeutic agent is doxorubicin, irinotecan, sunitinib, sorafenib, paclitaxel, temozolomide, oxaliplatin, gemcitabine, carmustine, cyclophosphamide, vinchristine, and/or an antibody.

The liquid embolic formulation can be formulated as a solution and delivered in a syringe or vial. In other embodiments, the formulation can be prepared as a dry powder or lyophilate that needs to be reconstituted prior to use. In some embodiments, the pharmaceutical drug or therapeutic agent can be added to the liquid embolic prior to use or can be formulated into the liquid embolic when formed.

In some embodiments, then formulated in solution, the liquid embolic can be mixed with a pharmaceutical drug or therapeutic agent in a vial or syringe. The pharmaceutical drug or therapeutic agent can be a liquid or powder than needs reconstitution.

In some embodiments, the liquid embolic formulation can be removed from a vial using a needle and syringe. To prevent premature liquid embolic polymer deposition, the delivery catheter is flushed with a bolus of the same water miscible solvent as was used to dissolve the liquid embolic polymer. This flushing prevents clogging of the delivery catheter with the liquid embolic polymer. The syringe containing the liquid embolic formulation is then connected to the proximal end of delivery catheter, such as a microcatheter, cannula, or the like, positioned in the desired vascular or other anatomic site.

As the liquid embolic formulation is injected, it pushes the water miscible solvent flushing solution out of the microcatheter. The progress of the liquid embolic formulation inside the delivery catheter can be observed using an imaging technique compatible with the visualization species selected. With continued injection, the liquid embolic formulation can enter the target delivery site.

The solidified liquid embolic polymer can provide long-term occlusion of the target site. Over time, the biodegradable linkages binding the visualization species to the liquid embolic polymer are broken and the visualization of the liquid embolic polymer is diminished.

Further, the solidified liquid embolic polymer can provide delivery of the pharmaceutical drug or therapeutic agent to the target site. Over time, the pharmaceutical drug or therapeutic agent can elute from the liquid embolic polymer as described herein.

In some embodiments, the herein described formulations can be used to treat cancer.

In some embodiments, the herein described formulations can be used to treat a tumor.

In some embodiments, the herein described formulations can be used to treat an unwanted growth.

In some embodiments, the herein described formulations can be used to treat a proliferation of tissue.

### EXAMPLE 1

### Preparation of an Iodine-Containing Monomer

To 250 milliliters of toluene, 15 g triiodophenol, 22.9 g 3,6-dimethyl-1,4dioxane-2,5dione, and 25 microliters of stannous octoate are added. The solution is refluxed for 18 hr. After cooling the solution to 25° C., 3 mL acryloyl chloride and 5.2 mL triethylamine dissolved in 50 mL toluene are added. The mixture is stirred for 5 hr, filtered, washed with water, and dried under vacuum.

### EXAMPLE 2

### Preparation of an Iodine-Containing Polymer

To 3 milliliters of dimethyl sulfoxide, 1.8 g triiodophenol chain extended with an average of 5 lactide units and capped with an acrylate, 0.2 g of hydroxyethyl methacrylate, and 10 mg of azobisisobutyronitrile are added. Upon complete dissolution of all components, the solution is placed at 80° C. for 4 hours. After cooling to room temperature, the polymer is recovered by precipitation in ethyl ether and dried under vacuum.

### EXAMPLE 3

### Exchanging Iodine on an Iodine-Containing Polymer

To a dimethyl sulfoxide solution of the iodine-containing polymer of Example 2, Na¹³¹I is added with stirring. After the Na¹³¹I is completely dissolved, hydrogen peroxide (30% in aqueous solution) is added. The reaction is optionally heated to facilitate the exchange process. After 10 min of reaction time (or longer as needed), the DMSO solution is poured over distilled water to precipitate the iodine-containing polymer. The precipitate is filtered and subsequently redissolved in DMSO and reprecipitated in DI water twice more. The solid is then lyophilized to remove water and obtain the product as a solid.

### EXAMPLE 4

### Preparation of Liquid Embolic Formulation

To 9 g of dimethyl sulfoxide, one gram of the polymer of Example 3 is added. The liquid embolic formulation is then aliquoted into vials and capped. The vials are autoclaved at 121° C. for 15 minutes.

### EXAMPLE 5

### In Vitro Elution of Pharmaceutical Agents

Fifty mg of paclitaxel are dissolved in 1 mL of 25 wt % PHIL® solution, which includes triiodophenol-(lactide-co-glycolide) acrylate and hydroxyethyl methacrylate in dimethyl sulfoxide. The 1 mL of 25 wt % PHIL®/Paclitaxel solution is precipitated in 199 mL of a dissolution medium, consisting of 45:55 acetonitrile/10 mM potassium phosphate buffer solution pH 4.5 at room temperature. At 2, 5, 9, 14, 20, 27, 35, 44, 54, 65 and 90 minutes, 1 mL of the supernatant is pipetted out and placed in a HPLC vial.

The concentration of paclitaxel in each sample is determined using an Agilent 1100 HPLC system. The chromatographic analysis is performed with an Agilent Extended-C18 column (4.6 mm×50 mm, 3.5 μm). The mobile phase includes 50% acetonitrile and 50% 5% acetonitrile in water delivered at 1 mL/min. The injection volume is 10 μL and the wavelength of the ultraviolet detector was 227 nm. The calibration curve is prepared from 5 to 500 ppm of paclitaxel. The amount of paclitaxel released and relative percentage are calculated from the concentration data.

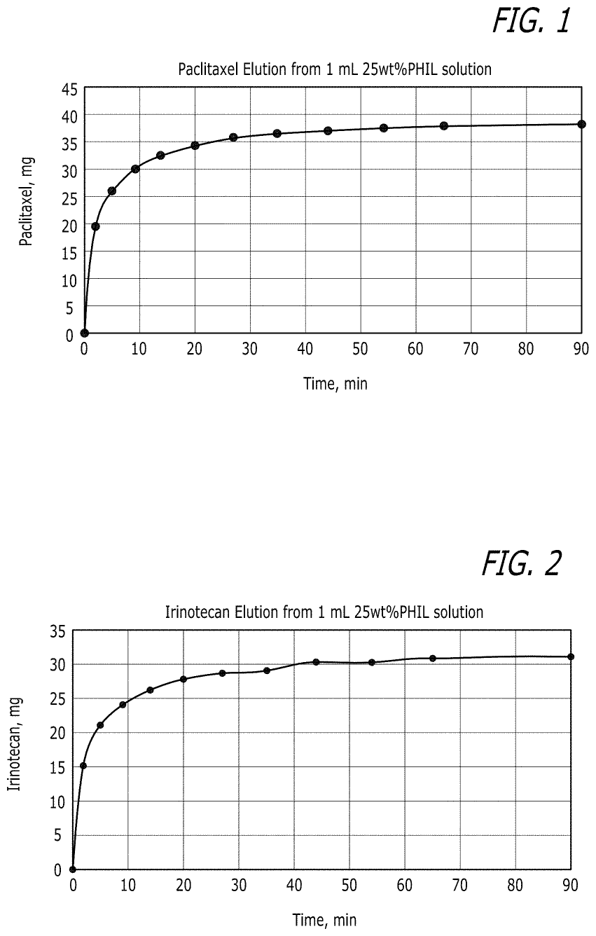

The kinetics of paclitaxel elution from the PHIL® solution are shown in FIG. 1. The elution curve obtained is close to a logarithmic curve over the period of 90 minutes with a sharp increase of 30 mg within the first 9 minutes before eventually plateauing through the 90 minutes period. The total amount of paclitaxel eluted during the first 90 minutes was 38 mg per 1 mL of PHIL®.

### EXAMPLE 6

### In Vitro Elution of Pharmaceutical Agents

Fifty mg of irinotecan hydrochloride are dissolved in 1 mL of 25 wt % PHIL® solution, which includes triiodophenol-(lactide-co-glycolide) acrylate and hydroxyethyl methacrylate in dimethyl sulfoxide. The 1 mL of 25 wt % PHIL®/irinotecan solution is precipitated in 99 mL of a dissolution medium, consisting of 10 mM potassium phosphate buffer solution pH 4.0 at room temperature. At 2, 5, 9, 14, 20, 27, 35, 44, 54, 65 and 90 minutes, 1 mL of the supernatant is pipetted out and placed in a HPLC vial.

The concentration of irinotecan in each sample is determined using an Agilent 1100 HPLC system. The chromatographic analysis is performed with an Agilent Extended-C18 column (4.6 mm×50 mm, 3.5 μm). The mobile phase includes of 18% acetonitrile and 82% 10 mM potassium phosphate buffer solution pH 3 with 5% acetonitrile and 7.2 mM triethylamine delivered at 1 mL/min. The injection volume is 2 μL and the wavelength of the ultraviolet detector is 223 nm. The calibration curve is prepared from 10 to 1000 ppm of irinotecan. The amount of irinotecan released and relative percentage are calculated from the concentration data.

The kinetics of irinotecan elution from the PHIL® solution are shown in FIG. 2. The elution curve obtained is close to a logarithmic curve over the period of 90 minutes with a sharp increase of 24 mg within the first 9 minutes before eventually plateauing through the 90 minutes period. The total amount of irinotecan eluted during the first 90 minutes is 31 mg per 1 mL of PHIL®.

### EXAMPLE 7

### In Vitro Elution of Pharmaceutical Agents

Fifty mg of doxorubicin hydrochloride are dissolved in 1 mL of 25 wt % PHIL® solution, which includes triiodophenol-(lactide-co-glycolide) acrylate and hydroxyethyl methacrylate in dimethyl sulfoxide. The 1 mL of 25 wt % PHIL®/doxorubicin solution is precipitated in 99 mL of a dissolution medium, including 10 mM potassium phosphate buffer solution pH 4.0 at room temperature. At 2, 5, 9, 14, 20, 27, 35, 44, 54, 65 and 90 minutes, 1 mL of the supernatant is pipetted out and placed in a HPLC vial.

The concentration of doxorubicin in each sample is determined using an Agilent 1100 HPLC system. The chromatographic analysis is performed with an Agilent Extended-C18 column (4.6 mm×50 mm, 3.5 μm). The mobile phase includes 18% acetonitrile and 82% 10 mM potassium phosphate buffer solution pH 3 with 5% acetonitrile and 7.2 mM triethylamine delivered at 1 mL/min. The injection volume is 3 μL and the wavelength of the ultraviolet detector is 234 nm. The calibration curve is prepared from 10 to 1000 ppm of doxorubicin. The amount of doxorubicin released and relative percentage are calculated from the concentration data.

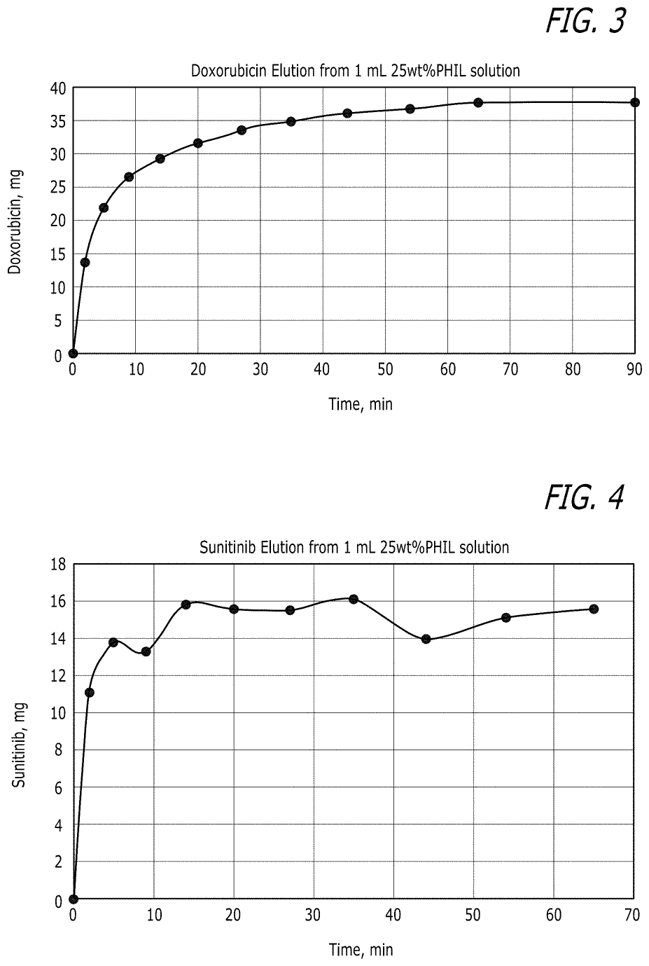

The kinetics of doxorubicin elution from the PHIL® solution are shown in FIG. 3. The elution curve obtained is close to a logarithmic curve over the period of 90 minutes with a sharp increase of 29 mg within the first 14 minutes before eventually plateauing through the 90 minutes period. The total amount of doxorubicin eluted during the first 90 minutes is 38 mg per 1 mL of PHIL®.

### EXAMPLE 8

### In Vitro Elution of Pharmaceutical Agents

Fifty mg of sunitinib malate are dissolved in 1 mL of 25 wt % PHIL® solution, which includes triiodophenol-(lactide-co-glycolide) acrylate and hydroxyethyl methacrylate in dimethyl sulfoxide. The 1 mL of 25 wt % PHIL®/sunitinib solution is precipitated in 999 mL of a dissolution medium, including phosphate buffered saline. At 2, 5, 9, 14, 20, 27, 35, 44, 54 and 65 minutes, 1 mL of the supernatant is pipetted out and placed in a HPLC vial.

The concentration of sunitinib in each sample is determined using an Agilent 1100 HPLC system. The chromatographic analysis is performed with an Agilent Extended-C18 column (4.6 mm×50 mm, 3.5 μm). The mobile phase includes 22% acetonitrile and 78% 10 mM potassium phosphate buffer solution pH 3 with 5% acetonitrile and 7.2 mM triethylamine delivered at 1 mL/min. The injection volume is 3 μL and the wavelength of the ultraviolet detector is 429 nm. The calibration curve is prepared from 1 to 100 ppm of sunitinib. The amount of sunitinib released and relative percentage are calculated from the concentration data.

The kinetics of sunitinib elution from the PHIL® solution are shown in FIG. 4. The elution curve obtained is close to a logarithmic curve over the period of 65 minutes with a sharp increase of 11 mg within the first 2 minutes before eventually plateauing through the 65 minutes period. The total amount of sunitinib eluted during the first 65 minutes is 16 mg per 1 mL of PHIL®.

### EXAMPLE 9

### In Vitro Elution of Pharmaceutical Agents

Fifty mg of sorafenib are dissolved in 1 mL of 25 wt % PHIL® solution, which includes triiodophenol-(lactide-co-glycolide) acrylate and hydroxyethyl methacrylate in dimethyl sulfoxide. The 1 mL of 25 wt % PHIL®/sorafenib solution is precipitated in 999 mL of a dissolution medium, including 70:30 acetonitrile/10 mM potassium phosphate buffer solution pH 4.3 at room temperature. At 2, 5, 9, 14, 20, 27, 35, 44, 54, 65 and 90 minutes, 1 mL of the supernatant is pipetted out and placed in a HPLC vial.

The concentration of sorafenib in each sample is determined using an Agilent 1100 HPLC system. The chromatographic analysis is performed with an Agilent Extended-C18 column (4.6 mm×50 mm, 3.5 μm). The mobile phase includes 50% acetonitrile and 50% 10 mM potassium phosphate buffer solution pH 3 with 5% acetonitrile and 7.2 mM triethylamine delivered at 1 mL/min. The injection volume is 3 μL and the wavelength of the ultraviolet detector is 263 nm. The calibration curve is prepared from 1 to 100 ppm of sorafenib. The amount of sorafenib released and relative percentage are calculated from the concentration data.

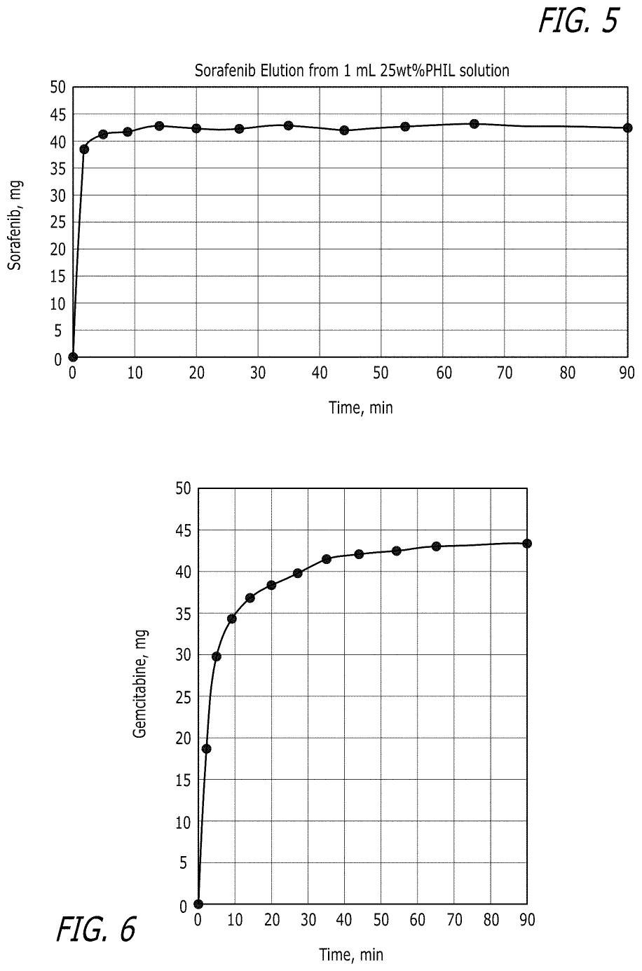

The kinetics of sorafenib elution from the PHIL® solution are shown in FIG. 5. The elution curve obtained is close to a logarithmic curve over the period of 90 minutes with a sharp increase of 38 mg within the first 2 minutes before eventually plateauing through the 90 minutes period. The total amount of sorafenib eluted during the first 90 minutes is 42 mg per 1 mL of PHIL®.

### EXAMPLE 10

### In Vitro Elution of Pharmaceutical Agents

Fifty mg of gemcitabine are dissolved in 1 mL of 25 wt % PHIL® solution, which includes triiodophenol-(lactide-co-glycolide) acrylate and hydroxyethyl methacrylate in dimethyl sulfoxide. The 1 mL of 25 wt % PHIL®/gemcitabine solution is precipitated in 99 mL of a dissolution medium, including phosphate buffered saline (PBS) at room temperature. At 2, 5, 9, 14, 20, 27, 35, 44, 54, 65 and 90 minutes, 1 mL of the supernatant is pipetted out and placed in a HPLC vial.

The concentration of gemcitabine in each sample is determined using an Agilent 1100 HPLC system. The chromatographic analysis is performed with an Agilent Extended-C18 column (4.6 mm×50 mm, 3.5 μm). The mobile phase includes HPLC water with 5% acetonitrile delivered at 1 mL/min. The injection volume is 3 μL and the wavelength of the ultraviolet detector is 275 nm. The calibration curve is prepared from 10 to 1000 ppm of gemcitabine. The amount of gemcitabine released and relative percentage are calculated from the concentration data.

The kinetics of gemcitabine elution from the PHIL® solution are shown in FIG. 6. The elution curve obtained is close to a logarithmic curve over the period of 90 minutes with a sharp increase of 34 mg within the first 9 minutes before eventually plateauing through the 90 minutes period. The total amount of gemcitabine eluted during the first 90 minutes is 43 mg per 1 mL of PHIL®.

### EXAMPLE 11

### In Vitro Elution of Pharmaceutical Agents

Fifty mg of oxaliplatin are dissolved in 1 mL of 25 wt % PHIL® solution, which includes triiodophenol-(lactide-co-glycolide) acrylate and hydroxyethyl methacrylate in dimethyl sulfoxide. The 1 mL of 25 wt % PHIL®/oxaliplatin solution is precipitated in 50 mL of a dissolution medium, including phosphate buffered saline (PBS) at room temperature. At 4.5, 15, 24.5, 34.5, 47.5, 60 and 80 minutes, 10 mL of the supernatant is pipetted out and placed in a 15 mL centrifuge tube. The remaining supernatant is poured out and the precipitates are mixed with a fresh 50 mL of the dissolution medium at room temperature.

Samples are prepared for ICP-MS analysis to measure the platinum concentration in the supernatant by mixing a sample portion (1 mL) with 4 mL of 2% nitric acid (5-fold dilution) or a sample portion (2.5 mL) with 0.05 mL of 2% nitric acid (undiluted). The calibration curve is prepared from 0.5 to 100 ppm of platinum. The amount of platinum released and relative percentage are calculated from the concentration data.

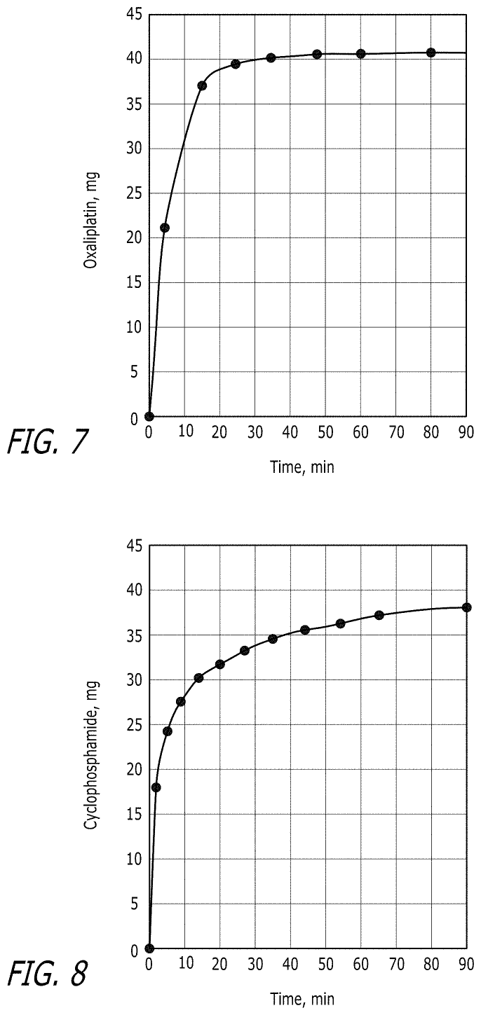

The kinetics of oxaliplatin elution from the PHIL® solution are shown in FIG. 7. The elution curve obtained is close to a logarithmic curve over the period of 80 minutes with a sharp increase of 37 mg within the first 15 minutes before eventually plateauing through the 34.5 minutes period. The total amount of oxaliplatin eluted during the first 80 minutes is 41 mg per 1 mL of PHIL®.

### EXAMPLE 12

### In Vitro Elution of Pharmaceutical Agents

Fifty mg of cyclophosphamide are dissolved in 1 mL of 25 wt % PHIL® solution, which includes triiodophenol-(lactide-co-glycolide) acrylate and hydroxyethyl methacrylate in dimethyl sulfoxide. The 1 mL of 25 wt % PHIL®/cyclophosphamide solution is precipitated in 99 mL of a dissolution medium, including distilled water at room temperature. At 2, 5, 9, 14, 20, 27, 35, 44, 54, 65 and 90 minutes, 1 mL of the supernatant is pipetted out and placed in a HPLC vial.

The concentration of cyclophosphamide in each sample is determined using an Agilent 1100 HPLC system. The chromatographic analysis is performed with a Primesep 100 column (3.2 mm×50 mm, 3 μm). The mobile phase includes 5% acetonitrile and 95% HPLC water with 5% acetonitrile delivered at 1 mL/min. The injection volume is 25 μL and the wavelength of the ultraviolet detector is 197 nm. The calibration curve is prepared from 10 to 1000 ppm of cyclophosphamide. The amount of cyclophosphamide released and relative percentage are calculated from the concentration data.

The kinetics of cyclophosphamide elution from the PHIL® solution are shown in FIG. 8. The elution curve obtained is close to a logarithmic curve over the period of 90 minutes with a sharp increase of 35 mg within the first 35 minutes before eventually plateauing through the 90 minutes period. The total amount of cyclophosphamide eluted during the first 90 minutes is 38 mg per 1 mL of PHIL®.

### EXAMPLE 13

### In Vitro Elution of Pharmaceutical Agents

Fifty mg of temozolomide are dissolved in 1 mL of 25 wt % PHIL® solution, which includes triiodophenol-(lactide-co-glycolide) acrylate and hydroxyethyl methacrylate in dimethyl sulfoxide. The 1 mL of 25 wt % PHIL®/temozolomide solution is precipitated in 99 mL of a dissolution medium, including HLPC water with 0.5% acetic acid at room temperature. At 2, 5, 9, 14, 20, 27, 35, 44, 54, 65 and 90 minutes, 1 mL of the supernatant is pipetted out and placed in a HPLC vial.

The concentration of temozolomide in each sample is determined using an Agilent 1100 HPLC system. The chromatographic analysis is performed with an Agilent Extended-C18 column (4.6 mm×50 mm, 3.5 μm). The mobile phase includes 10% methanol and 90% HPLC water with 0.5% acetic acid delivered at 1 mL/min. The injection volume is 3 μL and the wavelength of the ultraviolet detector is 330 nm. The calibration curve is prepared from 10 to 1000 ppm of temozolomide. The amount of temozolomide released and relative percentage are calculated from the concentration data.

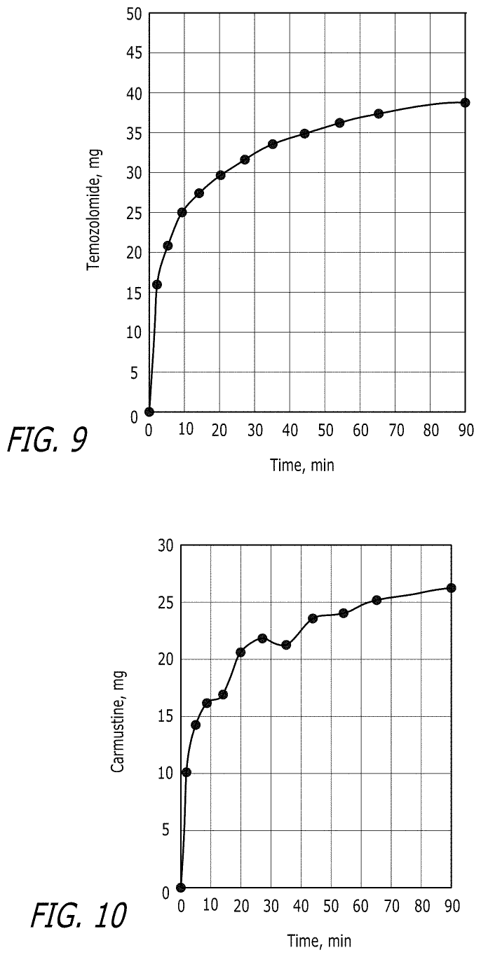

The kinetics of temozolomide elution from the PHIL® solution are shown in FIG. 9. The elution curve obtained is close to a logarithmic curve over the period of 90 minutes with a sharp increase of 34 mg within the first 35 minutes before eventually plateauing through the 90 minutes period. The total amount of temozolomide eluted during the first 90 minutes is 39 mg per 1 mL of PHIL®.

### EXAMPLE 14

### In Vitro Elution of Pharmaceutical Agents

Fifty mg of carmustine are dissolved in 1 mL of 25 wt % PHIL® solution, which includes triiodophenol-(lactide-co-glycolide) acrylate and hydroxyethyl methacrylate in dimethyl sulfoxide. The 1 mL of 25 wt % PHIL®/carmustine solution is precipitated in 99 mL of a dissolution medium, including distilled water at room temperature. At 2, 5, 9, 14, 20, 27, 35, 44, 54, 65 and 90 minutes, 1 mL of the supernatant is pipetted out and placed in a HPLC vial.

The concentration of carmustine in each sample is determined using an Agilent 1100 HPLC system. The chromatographic analysis is performed with a Primesep 100 column (3.2 mm×50 mm, 3 μm). The mobile phase includes 10% methanol and 90% 10 mM potassium phosphate buffer solution pH 3 with 5% acetonitrile and 7.2 mM triethylamine delivered at 1 mL/min. The injection volume is 3 μL and the wavelength of the ultraviolet detector is 230 nm. The calibration curve is prepared from 10 to 1000 ppm of carmustine. The amount of carmustine released and relative percentage are calculated from the concentration data.

The kinetics of carmustine elution from the PHIL® solution are shown in FIG. 10. The elution curve obtained is close to a logarithmic curve over the period of 90 minutes with a sharp increase of 21 mg within the first 35 minutes before eventually plateauing through the 90 minutes period. The total amount of carmustine eluted during the first 90 minutes is 26 mg per 1 mL of PHIL®.

### EXAMPLE 15

### In Vivo Evaluation of PHIL LV Liquid Embolic With Irinotecan in Canine Liver

A canine is anesthetized and a 6 Fr sheath is inserted into the femoral artery via a cutdown. A 6 Fr Glidecath is advanced retrograde into the celiac trunk and further into the hepatic artery. Following angiography, a Scepter balloon (4 mm×10 mm) is advanced through the Glidecath and into a branch of the hepatic artery. The balloon is inflated and 1.5 mL PHIL LV loaded with 75 mg irinotecan is injected into the hepatic artery branch. Blood is collected at 5, 15, 30, 60, and 120 minutes post-embolization for irinotecan quantification.

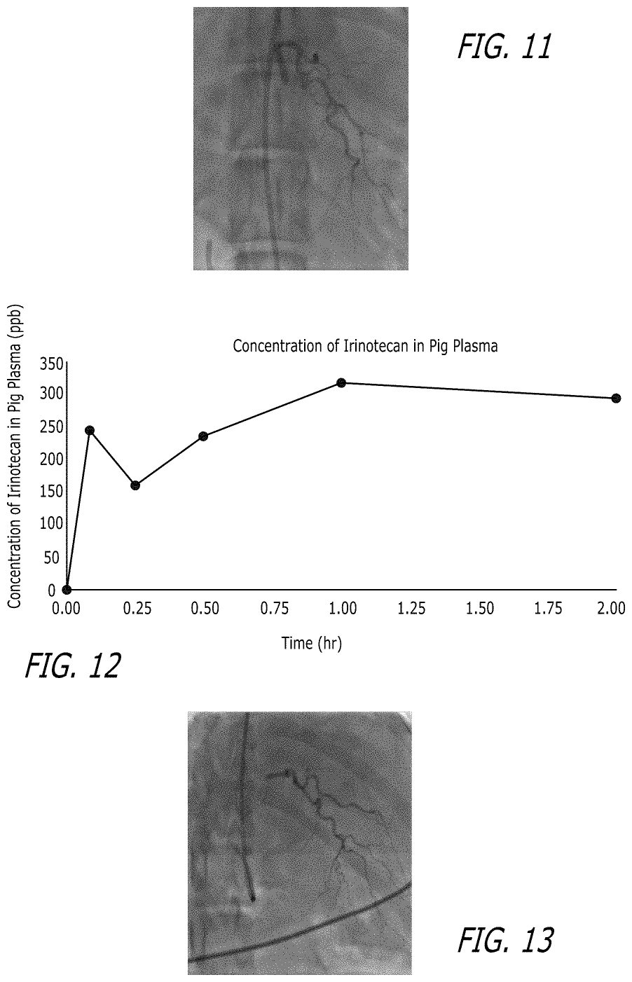

Post-embolization, angiography illustrated in FIG. 11 demonstrates excellent penetration into the distal liver vasculature. Reflux into other branches of the liver vasculature is not observed.

The quantification of irinotecan in the blood shows a rapid rise to about 300 ppb with any tapering back to baseline beyond the timescale of the experiment, as shown in FIG. 12.

### EXAMPLE 16

### In Vivo Evaluation of PHIL 25 Liquid Embolic With Doxorubicin in Canine Liver

A canine is anesthetized and a 6 Fr sheath is inserted into the femoral artery via a cutdown. A 6 Fr Glidecath is advanced retrograde into the celiac trunk and further into the hepatic artery. Following angiography, a Scepter balloon (4 mm×10 mm) is advanced through the Glidecath and into a branch of the hepatic artery. The balloon is inflated and 1.6 mL PHIL 25 loaded with 80 mg doxorubicin is injected into the hepatic artery branch. Blood is collected at 5, 15, 30, 60, and 120 minutes post-embolization for doxorubicin quantification.

Post-embolization, angiography as illustrated in FIG. 13 demonstrates excellent penetration into the distal liver vasculature. Reflux into other branches of the liver vasculature is not observed.

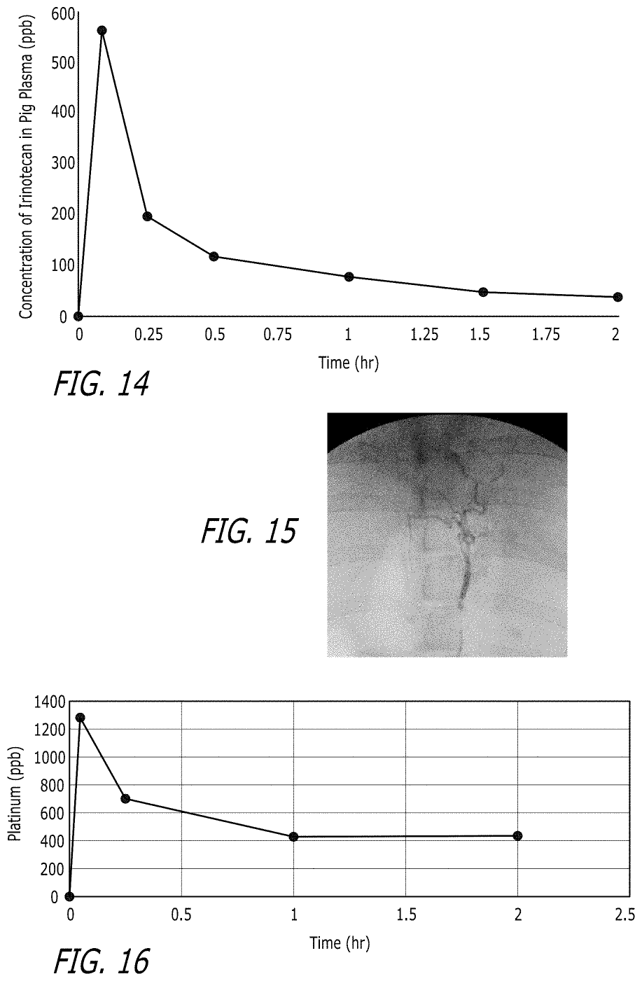

The quantification of doxorubicin in the blood shows a rapid rise to about 600 ppb in five minutes and tapering back to baseline over 2 hours, as shown in FIG. 14.

### EXAMPLE 17

### In Vivo Evaluation of PHIL LV Liquid Embolic With Oxaliplatin in Porcine Liver

A pig is anesthetized and a 6 Fr sheath is inserted into the femoral artery via a cutdown. A 6 Fr Glidecath is advanced retrograde into the celiac trunk and further into the hepatic artery. Following angiography, a Scepter balloon (4 mm×10 mm) is advanced through the Glidecath and into a branch of the hepatic artery. The balloon is inflated and 1.5 mL PHIL LV loaded with 30 mg oxaliplatin is injected into the hepatic artery branch. Blood is collected at 5, 15, 30, 60, and 120 minutes post-embolization for doxorubicin quantification.

Post-embolization, angiography as illustrated in FIG. 15 demonstrates excellent penetration into the distal liver vasculature. Reflux into other branches of the liver vasculature is not observed.

The quantification of oxaliplatin in the blood shows a rapid rise to about 1,300 ppb in five minutes and tapering back to baseline beyond the time scale of the experiment, as shown in FIG. 16.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

We claim:

1. An embolic composition comprising:
a substantially stable biocompatible polymer comprising a reaction product of a first monomer including a polymerizable moiety having a biodegradable or biostable linkage to a visualization agent having at least one aromatic ring including at least one iodine atom and a second monomer including a polymerizable moiety and at least one hydroxyl group; and
a non-physiological solution containing a pharmaceutical drug or therapeutic agent;
wherein the substantially stable biocompatible polymer is soluble in the non-physiological solution and insoluble in a physiological solution.

2. The embolic composition of claim 1, wherein at least one of the at least one iodine atom is a radioactive isotope.

3. The embolic composition of claim 2, wherein the radioactive isotope is ¹²³I, ¹²⁴I, ¹²⁵I, ¹³¹I, or a combination thereof.

4. The embolic composition of claim 1, wherein the pharmaceutical drug or therapeutic agent is doxorubicin, irinotecan, sunitinib, sorafenib, paclitaxel, temozolomide, oxaliplatin, gemcitabine, carmustine, cyclophosphamide, vinchristine, an antibody, or a combination thereof.

5. The embolic composition of claim 1, wherein the pharmaceutical drug or therapeutic agent is paclitaxel.

6. The embolic composition of claim 1, wherein the pharmaceutical drug or therapeutic agent is irinotecan.

7. The embolic composition of claim 1, wherein the pharmaceutical drug or therapeutic agent is doxorubicin.

8. The embolic composition of claim 1, wherein the pharmaceutical drug or therapeutic agent is sunitinib.

9. The embolic composition of claim 1, wherein the pharmaceutical drug or therapeutic agent is sorafenib.

10. The embolic composition of claim 1, wherein the pharmaceutical drug or therapeutic agent is gemcitabine.

11. The embolic composition of claim 1, wherein the pharmaceutical drug or therapeutic agent is oxaliplatin.

12. The embolic composition of claim 1, wherein the pharmaceutical drug or therapeutic agent is cyclophosphamide.

13. The embolic composition of claim 1, wherein the pharmaceutical drug or therapeutic agent is temozolomide.

14. The embolic composition of claim 1, wherein the pharmaceutical drug or therapeutic agent is carmustine

15. A method of treatment, the method comprising:
delivering to a treatment site an embolic composition including
a substantially stable biocompatible polymer comprising a reaction product of a first monomer including a polymerizable moiety having a biodegradable or biostable linkage to a visualization agent having at least one aromatic ring including at least one iodine atom and a second monomer including a polymerizable moiety and at least one hydroxyl group; and
a non-physiological solution containing a pharmaceutical drug or therapeutic agent;
wherein the substantially stable biocompatible polymer is soluble in the non-physiological solution and insoluble in a physiological solution,
treating a condition present at the treatment site.

16. The method of claim 15, wherein the delivering results in the substantially stable biocompatible polymer precipitating in the physiological solution.

17. The method of claim 15, wherein the treatment site is within a lumen.

18. The method of claim 15, wherein the condition is a cancer, a tumor, an unwanted growth, a proliferation of tissue, or a combination thereof.

19. The method of claim 15, wherein the delivering results in an elution of the pharmaceutical drug or therapeutic agent.

20. The method of claim 19, wherein the elution is logarithmic.
